(19) United States
(12) Patent Application Publication
(10) Pub. No.: US 2022/0184254 A1
(43) Pub. Date: Jun. 16, 2022

(54) Ultraviolet Light Sanitizer

(71) Applicant: Heather Zack, Parma, OH (US)

(72) Inventor: Heather Zack, Parma, OH (US)

(21) Appl. No.: 17/549,953

(22) Filed: Dec. 14, 2021

Related U.S. Application Data
(60) Provisional application No. 63/125,059, filed on Dec. 14, 2020.

(51) Int. Cl. A61L 2/10; A61L 2/24

(52) U.S. Cl. CPC A61L 2/10; A61L 2/24; A61L 2202/11; A61L 2202/25; A61L 2202/123; A61L 2202/14; A61L 2202/16; A61L 2202/121

(57) ABSTRACT

A multipurpose ultraviolet light sanitizing device is disclosed. The novel sanitizing device comprises one or more ultraviolet radiation sources to radiate ultraviolet light for sanitization purposes, an adhesive tape or other attachment means on the rear surface of the device for mounting the light device to any surface, and a wireless communication module for making a connection with a remote device. The ultraviolet light device comes in different variants such as in the form of a single disinfectant light device or in the form of a strip having a plurality of ultraviolet lights positioned there along. The novel ultraviolet light of the present invention helps to eliminate germs, viruses and bacteria, thereby creating a safe and sanitary environment.

### CROSS-REFERENCE TO RELATED APPLICATION

The present application claims priority to, and the benefit of, U.S. Provisional Application No. 63/125,059, which was filed on Dec. 14, 2020 and is incorporated herein by reference in its entirety.

### FIELD OF THE INVENTION

The present invention relates generally to the field of sanitization. More specifically, the present invention relates to a multipurpose ultraviolet (UV) light system that eliminates germs, viruses, bacteria and other harmful pathogens, thereby creating a safe and sanitary environment for the users. The multipurpose ultraviolet light system comprises one or more ultraviolet lights attached to a strip featuring an adhesive on its back portion. The multipurpose ultraviolet light system is capable of being installed in vehicles, such as cars, trains, buses, airplanes, etc., homes, offices or any other areas requiring sanitization. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, various harmful pathogens, germs, bacteria, viruses and the like are present in the environment, and are responsible for the spread of infectious diseases amongst people. More specifically, such harmful pathogens can easily transmit from one person to another, and may cause serious illness and even death in critical cases. These harmful pathogens may be present on any surface touched by an infected person, which when touched by a healthy person, leads to the spread of diseases amongst individuals. When an infected person sneezes or coughs, or even touches a surface, the bacteria, viruses, and other disease-causing microbes adhere to said surfaces after contact. In this manner, it is believed that billions of individuals have spread infectious diseases among each other over the years including, without limitation, the common cold, influenza, rotavirus, hepatitis A, tuberculosis, conjunctivitis, staphylococcal bacterial infections, COVID-19, strep throat and other streptococcal bacterial infections.

Further, in the times of a pandemic, such as the ongoing COVID-19 pandemic which is caused by the exponential spread of disease-causing microbes, the transmission of the infectious microbes may be instantaneous, thereby causing the disease to rapidly spread across a large region, for instance throughout multiple continents or worldwide and affect a substantial number of individuals.

Various solutions have been proposed in the state of the art for the containment and mitigation of pandemics and other infectious diseases. For example, individuals are advised to take certain precautions at the personal level to maintain hygiene, and to protect themselves from the spread of harmful diseases. Personal preventive measures may include, without limitation, wearing face masks, washing hands frequently, sanitization of surfaces, self-quarantine, etc. However, many of such individuals may not always be able to adhere to such guidelines and preventative measures, and may forget to wear a face mask, avoid touching their face, wash their hands frequently, etc. Unfortunately, without taking such preventative measures, there is a high risk of the spread of disease amongst individuals.

Additionally, proper and preventative sanitization requires an individual to manually wipe and clean each and every surface that he or she comes into contact with a disinfecting or sanitizing solution. However, manually cleaning every such surface is time consuming, tiring, ineffective and may result in some spots being left unclean. This can lead to the spread of germs, bacteria and dangerous viruses. Also, with manual cleaning and sanitizing attempts, it is also difficult to ensure high-level disinfection due to a number of challenges including, but not limited to, missed areas, re-contamination from dirty sponges and mops, and improper usage and mixing of antibacterial cleaning solutions, etc.

Additionally, individuals who travel may use rental vehicles or modes of public transportation, each of which can also be a source of disease transmission. Further, individuals may lack the proper means to clean all areas inside a rental vehicle or other form of public transportation each and every time they enter or utilize the same. Dangerous pathogens may be present on the surfaces of such vehicles, which may easily be transmitted to the driver or passengers who may be present in the vehicle.

Therefore, there exists a long felt need in the art for a sanitization device that may be used to effectively and automatically disinfect contact surfaces as per the desires of the user, and that can eliminate the disease-causing microbes commonly found on frequently touched surfaces. Additionally, there is a long felt need in the art for a sanitizing device that can be easily and automatically controlled by the user, and that prevents the transmission of harmful pathogens and therefore prevents the spread of infectious diseases. Further, there is a long felt need in the art for a sanitizing device that requires little of the user's time or effort, and that ensures that all contact surfaces are treated, thereby preventing any inadvertent disease transmission. Moreover, there is a long felt need in the art for a sanitizing device that is both portable and relatively maintenance free. Finally, there is a long felt need in the art for a sanitizing device that is relatively inexpensive to manufacture and operate, and that is both safe and easy to use.

The subject matter disclosed and claimed herein, in one embodiment thereof, comprises a novel and multipurpose disinfecting ultraviolet light device to eliminate germs, viruses and bacteria. The ultraviolet light sanitizer comprises one or more ultraviolet radiation sources to radiate ultraviolet light to sanitize an object or a surface, an adhesive tape applied to the rear surface of the device or other means for mounting the novel light device to a surface, a wireless communication module for making a wireless connection with a remote control device wherein the control device pairs with the light device and controls the operation of the plurality of radiation sources, an internal battery to power the ultraviolet radiation sources and the wireless communication module and a timer circuit to set a timer function of the ultraviolet light sanitizer device.

In this manner, the novel sanitizer device of the present invention accomplishes all of the forgoing objectives, and provides a relatively convenient, efficient and automatic solution to disinfect and treat various contact surfaces and areas, thereby preventing the spread of disease-causing microbes. The ultraviolet light sanitizer of the present invention is also user friendly since it is portable and does not require the user to physically wipe and disinfect every contact surface, thereby saving time and effort.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a multipurpose disinfecting ultraviolet light device to eliminate germs, viruses and bacteria. The device comprises one or more ultraviolet radiation sources to radiate ultraviolet light for sanitization purposes, an adhesive tape applied to the rear surface of the device or other means for mounting the novel light device to any surface or object, a wireless communication module for making a wireless connection with a remote electronic device wherein the remote electronic device pairs with the novel light device and controls the operation of the radiation sources, an internal battery to power the ultraviolet radiation sources and a timer circuit to set a timer function in relation to the ultraviolet radiation sources.

In a further embodiment of the present invention, an ultraviolet disinfecting light device configured to emit ultraviolet radiation towards a surface to be treated is disclosed. The disinfecting light device comprises a single ultraviolet radiation source shaped in any geometric form that emits ultraviolet radiation from its front surface outwardly, an adhesive tape applied to the rear surface of the device, or other means for mounting the novel light device to a surface, a proximity sensor, a wireless communication module for creating a wireless communication channel, an internal battery to power the ultraviolet radiation source and a timer circuit to set a timer function in relation to the ultraviolet radiation source. The ultraviolet disinfecting light device can be controlled with a paired mobile application installed on an electronic device or an Internet of Things (IoT) hub/controller through a voice command or touch control.

In yet a further embodiment of the present invention, an ultraviolet disinfecting light device configured to emit ultraviolet radiation towards a potentially contaminated surface is disclosed. The device comprises a plurality of ultraviolet radiation sources in the form of a strip or a substrate wherein the strip or substrate is flexible, an adhesive tape applied to the rear surface of the strip/substrate for mounting the light device to any surface, including a curved surface, a proximity sensor to sense any motion near the device and trigger activation of the radiation source, a wireless communication module for creating a wireless communication channel with a remote control device, an internal battery to power the ultraviolet radiation sources and a timer circuit to set the timer function relative to the ultraviolet radiation sources, wherein the ultraviolet disinfecting light device connects to a smartphone application installed on the remote control device or an Internet of Things (IoT) hub/controller through the wireless communication channel.

In a further embodiment of the present invention, a method of disinfecting and sanitizing a confined space, such as a vehicle interior, home or business area, and/or a surface is disclosed. The method comprises the steps of (a) placing a novel sanitizing ultraviolet light device in a location that will maximize contact surface coverage of the confined space or surface for sanitization, (b) adding the sanitizing ultraviolet light device to an IoT network using a wireless communication module included with the sanitizing ultraviolet light device and (c) remotely activating and controlling the sanitizing ultraviolet light device through a wireless channel using an electronic device or IoT device.

The novel ultraviolet disinfecting light device of the various embodiments disclosed herein has a transparent or translucent component/cover placed over the ultraviolet radiation source to guide the ultraviolet light in various desired directions. The light device uses ultraviolet light to sterilize and decontaminate one or more surfaces. It should be appreciated that the ultraviolet disinfecting light device of the present invention is easy to use and can be installed virtually anywhere. The device can be used in homes, offices, vehicles and more. The disinfecting light is both portable and can be placed in close proximity to high-contact surfaces, such as door handles, light switches, steering wheels or any other suitable area, and may also be activated by motion or the lack thereof. The ultraviolet disinfecting light device of the present invention emits a germicidal light, such as an ultraviolet-C (UVC) wavelength. The ultraviolet disinfecting light device emits light in the wavelength range of 250-290 nm. In other embodiments, the preferred wavelength range is between 240-285 nm. The ultraviolet disinfecting light device can be used to disinfect the air, contact surfaces, and water present in the surrounding area.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the novel disinfecting ultraviolet light sanitizing device of the present invention in accordance with the disclosed architecture;

FIG. 2 illustrates a perspective view of another potential embodiment of the novel disinfecting ultraviolet light sanitizing device of the present invention in accordance with the disclosed architecture, wherein the device is in the form of a strip having a plurality of ultraviolet light devices positioned there along;

FIG. 3 illustrates a block diagram of the various components of one potential embodiment of the novel disinfecting ultraviolet light sanitizing device of the present invention in accordance with the disclosed architecture;

FIG. 4 illustrates a perspective view of another potential embodiment of the novel disinfecting ultraviolet light sanitizing device of the present invention in accordance with the disclosed architecture, wherein the device is positioned on the interior of a vehicle;

FIG. 5 illustrates a perspective view of another potential embodiment of the novel disinfecting ultraviolet light sanitizing device of the present invention in accordance with the disclosed architecture, wherein the device is positioned above a vehicular door handle;

FIG. 6 illustrates a perspective view of the novel disinfecting ultraviolet light sanitizing strip of FIG. 2 placed behind the steering wheel of a vehicle in accordance with the disclosed architecture;

FIG. 7 illustrates a perspective view of the novel disinfecting ultraviolet light sanitizing device of FIG. 1 and the novel disinfecting ultraviolet light sanitizing strip of FIG. 2 attached to a ceiling of an enclosed space in accordance with the disclosed architecture; and

FIG. 8 illustrates a perspective view of another potential embodiment of the novel disinfecting ultraviolet light sanitizing device of the present invention in accordance with the disclosed architecture, wherein the device is wirelessly connected with a remote electronic device and a smart IoT device.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a UV light sanitization device that may be used to effectively and automatically disinfect the air, contact surfaces, water, objects and the like, and that can eliminate the disease-causing microbes commonly found thereon. Additionally, there is a long felt need in the art for a UV light sanitizing device that can be easily and automatically controlled by the user, and that prevents the transmission of harmful pathogens and therefore prevents the spread of infectious diseases. Further, there is a long felt need in the art for a UV light sanitizing device that requires minimal time or effort to operate, and that ensures that all contact surfaces are treated, thereby preventing any inadvertent disease transmission. Moreover, there is a long felt need in the art for a UV light sanitizing device that is both portable and relatively maintenance free. Finally, there is a long felt need in the art for a UV light sanitizing device that is relatively inexpensive to manufacture and operate, and that is both safe and easy to use.

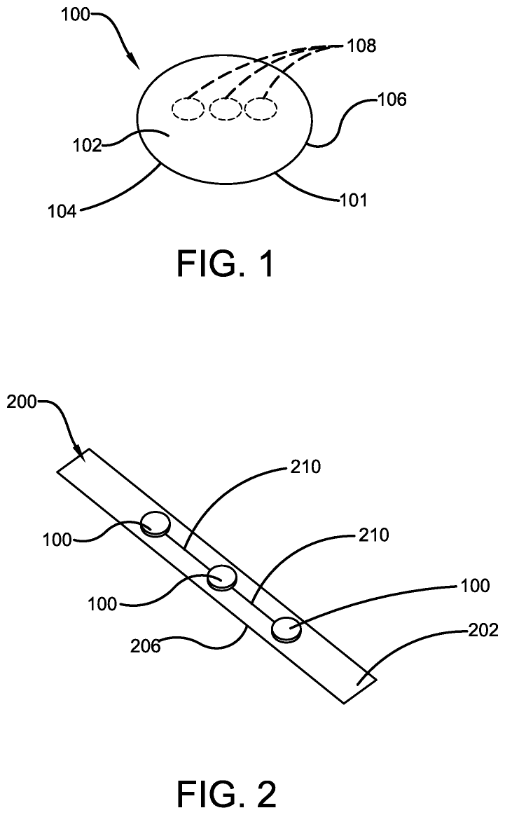

Referring initially to the drawings, FIG. 1 illustrates a perspective view of one potential embodiment of the novel disinfecting ultraviolet light sanitizing device 100 of the present invention in accordance with the disclosed architecture. As shown, the disinfecting ultraviolet light device 100 has a housing 101 and a plurality of ultraviolet radiating sources 108 contained therein that emit light at the proper wavelength suitable for sanitizing the surrounding area. The emitted germicidal light passes through the transparent or translucent cover 102 that further orients the emitted light in a specific direction for an efficient cleansing. The cover 102 is preferably sealed to the housing 101 around the periphery 104 and a rear surface 106 has an adhesive tape to mount the disinfecting ultraviolet light device 100 onto any surface or object. Alternatively, the rear surface 106 may include slotted holes for mounting the device 100 to a wall using one or more screws or other fasteners. The ultraviolet radiating sources 108 can be high intensity ultraviolet lamps, ultraviolet light emitting diodes (LEDs), super luminescent LEDs or a combination thereof.

The adhesive tape at the rear surface 106 allows the light device 100 to removably attach to a surface, and thus increases the utility of the light device 100. The portable sanitizing ultraviolet light device 100 provides effective protection from germs, viruses, bacteria, dust mites and mold without the use of aerosol sprays or other harsh chemicals that may be harmful if inhaled by an individual, particularly someone with a respiratory illness.

The disinfecting ultraviolet light device 100 may further include a vaporizable material, such as mercury. The mercury, when electrically activated, emits an ultraviolet light at a germicidal wavelength. As stated earlier, the ultraviolet light passes through the transparent or translucent cover 102. The ultraviolet light may be at least partially directed or refracted by the cover 102, which may serve as a lens. It should be appreciated that the disinfecting ultraviolet light device 100 may be of a toroidal, shape, unshaped, cylindrical, bulbous or any other shape.

FIG. 2 illustrates a perspective view of another potential embodiment of the novel disinfecting ultraviolet light sanitizing device 100 of the present invention in accordance with the disclosed architecture, wherein the device is in the form of a strip 200 having a plurality of ultraviolet light devices 100 positioned there along. As shown, a disinfecting ultraviolet light strip 200 is comprised of a plurality of the novel ultraviolet light devices 100 that are electrically connected 210 to each other, such that all of the individual ultraviolet light devices 100 may be illuminated in a single operation from a user. The ultraviolet light devices 100 may be placed on a flexible strip 202 comprising a plurality of electrical connections 210 that may be used to connect the plurality of ultraviolet light devices 100. Additionally, the rear or back surface 206 of the strip 202 may have an adhesive tape for mounting the ultraviolet light strip 200 onto any surface, regardless of its shape or curvature. The individual ultraviolet light devices 100 can be attached to the strip 202 by adhesive, rivets, stitching or any other suitable fastening means to form the disinfecting ultraviolet light strip 200. The disinfecting ultraviolet light strip 200 can also be manufactured in various different sizes, and can have any different number of individual ultraviolet light devices 100 attached thereto in a spaced-apart relationship, wherein each of the devices 100 may be electrically connected 210 together, or independently operable to suit user need and/or preference.

In another embodiment of the present invention, each of the individual ultraviolet light devices 100 of the disinfecting ultraviolet light strip 200 can be individually controlled by sending an instruction from the user through a remote device, such as a smartphone, tablet, smart watch, computer or the like, as explained more fully below. Once activated, each of the individual ultraviolet light devices 100 can emit germicidal wavelength light capable of disinfecting the surrounding air and contact surfaces. In fact, it should be noted that all the various embodiments of the disinfecting ultraviolet light (i.e., the disinfecting ultraviolet light strip 200 and the disinfecting ultraviolet light device 100) may be controlled wirelessly through a remote or electronic device. The electronic device may include a mobile application 810 (as best described in FIG. 8) that can control the ultraviolet disinfecting light. Additionally, if properly configured, the disinfecting ultraviolet lights 100, 200 can also be controlled through an IoT device.

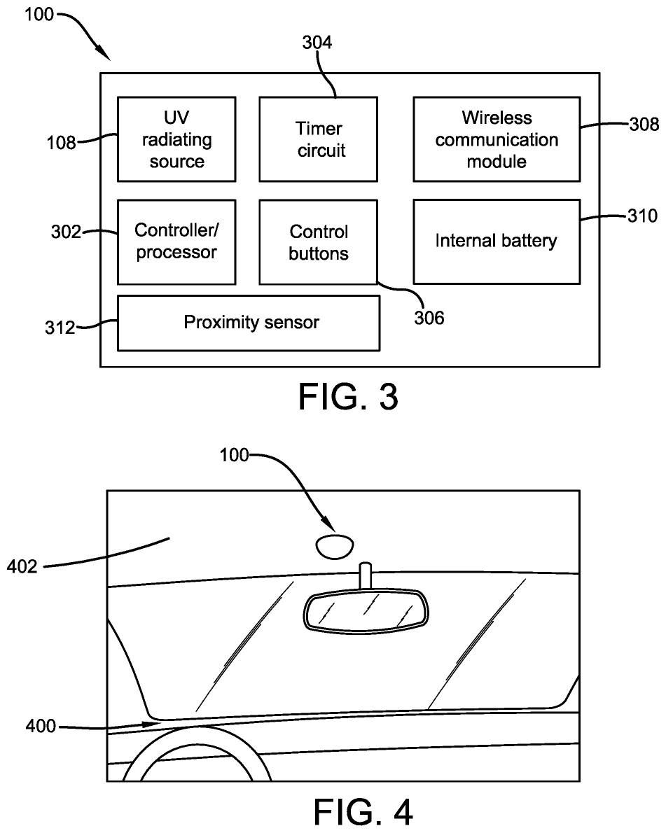

FIG. 3 illustrates a block diagram of the various components of one potential embodiment of the novel disinfecting ultraviolet light sanitizing device 100 of the present invention in accordance with the disclosed architecture. More specifically, the disinfecting ultraviolet light device 100 may comprise a controller/processor 302 that controls the operation of the ultraviolet radiating sources 108 as per instructions given by the user. The ultraviolet light device 100 also comprises a wireless communication module 308 that is used to create a wireless communication channel based on state-of-the-art wireless communication technology such as, but not limited to, Wi-Fi/Bluetooth/Zigbee/Z-Wave/RFID/NFC, for connecting to a smart device such as a smart switch, smart speaker, electronic device or the like. The wireless communication module 308 allows the user to operate the disinfecting ultraviolet light device 100 remotely, wherein the intensity, wavelength and other properties of the ultraviolet lights 108 can be changed remotely using the wireless communication channel.

In addition, the disinfecting ultraviolet light device 100 may also comprise a timer circuit 304 that can be used to turn the ultraviolet radiating sources 108 ON or OFF at specific times of the day or for specific time intervals. This feature is particularly useful in cases where the sanitization can be performed at a specific time when people are not present in the area. The timer circuit 304 can be programmed remotely as well. Further, the disinfecting ultraviolet light device 100 may also comprise one or more proximity sensors 312 so that the ultraviolet radiating sources 108 can be automatically turned OFF when motion is detected in the area. This safety feature protects any individual from inadvertently being in the surrounding area when the ultraviolet radiating sources 108 are turned ON.

Further, the disinfecting ultraviolet light device 100 includes an internal rechargeable battery 310 that is used to power the novel disinfecting ultraviolet light device 100. The ultraviolet radiating sources 108 can be turned ON by the control button 306 that may be located on the cover 102 or other suitable location, or through an instruction sent through a wireless channel via the wireless communication module 308. It should be appreciated that disinfecting ultraviolet light strip 200 can have individual electronic components for each disinfecting ultraviolet light 100, or alternatively can have centralized components to operate all of the individual disinfecting ultraviolet lights 100.

FIG. 4 illustrates a perspective view of another potential embodiment of the novel disinfecting ultraviolet light sanitizing device 100 of the present invention in accordance with the disclosed architecture, wherein the device 100 is positioned on the interior of a vehicle 400. As shown, the disinfecting ultraviolet light device 100 is mounted to the interior ceiling 402 of the vehicle 400 for the sterilization of the interior of the vehicle 400. The disinfecting ultraviolet light 100, based on instructions from the user through the wireless communication module 308, may emit germicidal wavelength light to disinfect the entire car interior. More specifically, the ceiling-mounted light device 100 sterilizes the surrounding air, seats, floor mats, dashboard and steering wheel simultaneously.

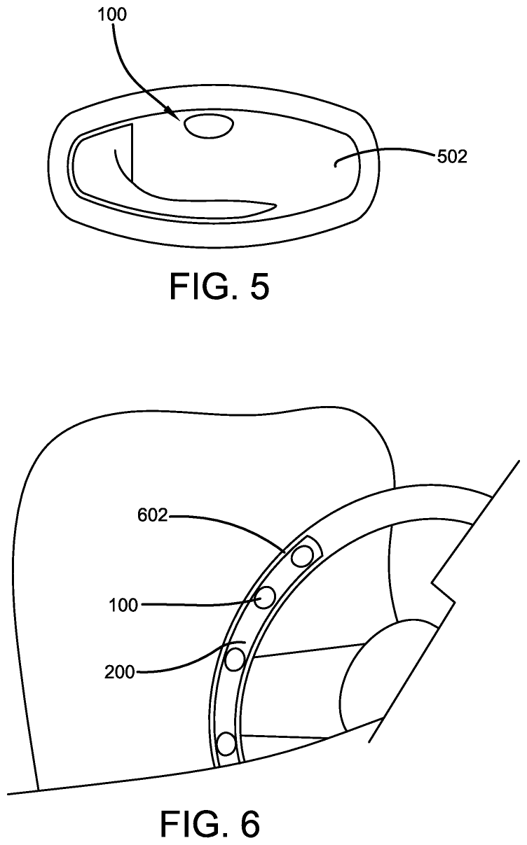

FIG. 5 illustrates a perspective view of another potential embodiment of the novel disinfecting ultraviolet light sanitizing device 100 of the present invention in accordance with the disclosed architecture, wherein the device 100 is positioned above a vehicular door handle 502. As shown, the disinfecting ultraviolet light device 100 is mounted above the vehicle door handle 502, as the handle 502 is typically one of the most highly contacted surfaces on the vehicle. This is especially true for vehicles that are shared by many people, such as is the case with rental vehicles. The light device 100 may be powered by battery 310, or may be hardwired into the exiting electrical system of the vehicle.

FIG. 6 illustrates a perspective view of the novel disinfecting ultraviolet light sanitizing strip 200 of FIG. 2 placed behind the steering wheel 602 of a vehicle in accordance with the disclosed architecture. More specifically, the disinfecting ultraviolet light strip 200 comprises a plurality of disinfecting ultraviolet light devices 100 along the length of the strip 202 which is relatively flexible and can be attached to the steering wheel 602 of the vehicle by an adhesive tape or other suitable attachment means, despite its curvature. It should be noted that the preferred size of the disinfecting lights 100 in the present embodiment is relatively small and conforming to the steering wheel 602 so that the driver of the vehicle does not feel the installed device while driving the car and using the steering wheel 602.

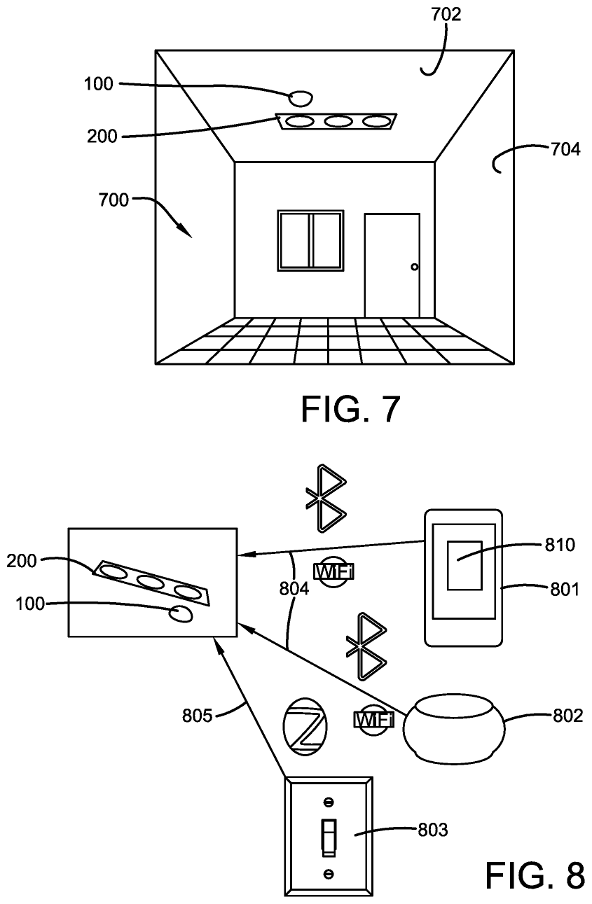

FIG. 7 illustrates a perspective view of the novel disinfecting ultraviolet light sanitizing device 100 of FIG. 1 and the novel disinfecting ultraviolet light sanitizing strip 200 of FIG. 2 attached to a ceiling 702 of an enclosed space or room 700 in accordance with the disclosed architecture. As shown, the combination of the novel disinfecting ultraviolet light 100 and the novel disinfecting ultraviolet light strip 200 are used in an interior space such as a room 700 in a home, office or other similar location to disinfect the surroundings and eliminate any disease-causing microbes, such as viruses, bacteria and other harmful pathogens. The disinfecting ultraviolet light 100 and/or the disinfecting ultraviolet light strip 200 can be secured to the ceiling surface using the adhesive tape on the backside of the lights 100 and 200, or alternatively, attached using screws or other fasteners. Based on the room-size and/or user requirements, one or more disinfecting ultraviolet lights 100 and disinfecting ultraviolet light strips 200 may be necessary to effectively disinfect the air and the contact surfaces of the room 700, thereby providing a sanitized environment for the users in the room 700.

Additionally, the position and placement of the lights 100, 200 may be changed as per the needs of the user. For instance, the device 100 and light strip 200 can also be secured on the walls 704 of the room 700 instead of, or in addition to, the ceiling 702. Further, the number and combination of devices 100 and strips 200 can be selected to best provide complete coverage of the room 700. Finally, one or more of the novel lights 100 and strips 200 can be individually turned ON or can be grouped together to turn ON or OFF at the same time because the wireless communication module of the device 100 may be in wireless communication with the wireless communication with the wireless communication module of the strip 200 to form a sanitization system.

FIG. 8 illustrates a perspective view of another potential embodiment of the novel disinfecting ultraviolet light sanitizing device 100 and light strip 200 of the present invention in accordance with the disclosed architecture, wherein the device/strip 100, 200 are wirelessly connected with a remote electronic device 801. As stated earlier, the disinfecting ultraviolet lights 100, 200 of the present invention each have a wireless communication module 308 for facilitating a wireless communication channel 804 or 805 that uses a wireless protocol such as Wi-Fi/Bluetooth/Zigbee/Wi-Fi Direct/RFID/Z-Wave. A smartphone 801, along with an installed companion mobile application 810, can control the ultraviolet light devices 100, 200. More specifically, the novel light devices 100, 200 can be easily and remotely turned ON or OFF through a smart home automation hub 802m such as Amazon Echo or Google Nest 802, via a user's voice command. Alternatively, through a different wireless protocol, such as Zigbee 805, the smart switch 803 can be connected to the novel light devices 100, 200 for controlling the operation of the same.

It should also be appreciated that the mobile application 810 used to control the novel ultraviolet lighting devices 100, 200 can be any form of software module installed on an electronic device or that can be accessed online through a web browser. The smartphone application 810 has the additional capability of changing the intensity, wavelength, color and other characteristics of the ultraviolet radiating sources 108. The smartphone application 810 can be downloaded from an online application store, and may be available for all mobile operating systems.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “disinfecting ultraviolet light device”, “disinfecting light device”, and “light device” are interchangeable and refer to the ultraviolet light sanitizing device 100 of the present invention.

Notwithstanding the forgoing, the ultraviolet light sanitizing device 100 of the present invention and its various components can be of any suitable size and configuration as is known in the art without affecting the overall concept of the invention, provided that it accomplishes the above stated objectives. One of ordinary skill in the art will appreciate that the size, configuration and material of the ultraviolet light sanitizing device 100 and its various components as shown in the FIGS. are for illustrative purposes only, and that many other sizes and shapes of the ultraviolet light sanitizing device 100 and the ultraviolet light strip 200 are well within the scope of the present disclosure. Although the dimensions of the light devices 100, 200 are important design parameters for user convenience, the light devices 100, 200 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A UV light sanitizing device comprising:
a housing;
at least one UV radiating source positioned in said housing;
a cover; and
a battery.

2. The UV light sanitizing device as recited in claim 1 further comprising a timer circuit.

3. The UV light sanitizing device as recited in claim 2 further comprising an adhesive position on a back surface of the housing.

4. The UV light sanitizing device as recited in claim 3 further comprising a wireless communication module.

5. The UV light sanitizing device as recited in claim 4, wherein the cover is a lens that directs the radiation emanating from the at least one UV radiating source in a particular direction.

6. The UV light sanitizing device as recited in claim 5, wherein the cover is comprised of a transparent material or a translucent material.

7. The UV light sanitizing device as recited in claim 5 further comprising a control button.

8. The UV light sanitizing device as recited in claim 7, wherein the at least one UV radiating source is in electrical communication with the battery.

9. The UV light sanitizing device as recited in claim 8, wherein the wireless communication module is in electrical communication with the battery.

10. The UV light sanitizing device as recited in claim 9, wherein the timer circuit is in electrical communication with the battery.

11. A UV light sanitizing device comprising:
a flexible substrate having a length, a front surface and a back surface;
a plurality of UV radiating sources positioned along said front surface in a spaced apart fashion;
an adhesive positioned along at least a portion of the back surface; and
a battery.

12. The UV light sanitizing device as recited in claim 11 further comprising a timer circuit.

13. The UV light sanitizing device as recited in claim 12 further comprising a wireless communication module.

14. The UV light sanitizing device as recited in claim 13, wherein each of the plurality of UV radiating sources comprises a cover.

15. The UV light sanitizing device as recited in claim 14, wherein the cover is a lens that directs the radiation emanating from the plurality of UV radiating sources in a particular direction.

16. The UV light sanitizing device as recited in claim 15, wherein the cover is comprised of a transparent material or a translucent material.

17. The UV light sanitizing device as recited in claim 16 further comprising a control button.

18. The UV light sanitizing device as recited in claim 17, wherein each of the plurality of UV radiating sources is in electrical communication with the battery.

19. The UV light sanitizing device as recited in claim 8, wherein each of the wireless communication module the timer circuit is in electrical communication with the battery.

20. A UV light sanitizing system comprising:
a flexible substrate having a length, a front surface, a back surface, a first wireless communication module, a battery and a plurality of first UV radiating sources positioned along said front surface in a spaced apart fashion; and
a UV light sanitizing device comprised of a housing containing at least one UV radiating source, a battery, a second wireless communication module and a cover, wherein the first wireless communication module is in wireless communication with the second wireless communication module.
